(19) United States
(12) Patent Application Publication
(10) Pub. No.: US 2026/0232451 A1
(43) Pub. Date: Aug. 13, 2026

(54) HYBRID REFERENCING DEVICE FOR KNEE ARTHROPLASTY

(71) Applicant: Michael C. Holmstrom, Cottonwood Heights, UT (US)

(72) Inventor: Michael C. Holmstrom, Cottonwood Heights, UT (US)

(21) Appl. No.: 19/537,340

(22) Filed: Feb. 11, 2026

Related U.S. Application Data
(60) Provisional application No. 63/757,644, filed on Feb. 12, 2025.

(51) Int. Cl. A61F 2/38; A61B 17/15; A61B 17/17; A61B 34/20; A61F 2/46

(52) U.S. Cl. CPC A61F 2/3859; A61B 17/155; A61B 17/1764; A61B 34/20; A61F 2/4657

(57) ABSTRACT

Hybrid-referencing devices, systems, methods, and techniques for knee arthroplasty are disclosed that include a use of both anterior and posterior landmarks for positioning a femoral component. Devices, systems methods and techniques are disclosed that can be used by a surgeon for sizing and positioning a femoral component in a knee arthroplasty, referencing both anterior and posterior landmarks in order to enhance or even optimize the procedure, including the capability to be used in both measured resection and/or gap balancing techniques.

### RELATED APPLICATIONS

The present application claims the benefit of priority under 35 U.S.C. § 119(e) to U.S. Provisional Patent Application 63/757,644, titled “UNIVERSAL FEMORAL SIZING AND POSITIONING DEVICE FOR KNEE ARTHROPLASTY,” filed Feb. 12, 2025, which is hereby incorporated herein by reference in its entirety.

### TECHNICAL FIELD

The present disclosure relates generally to the field of knee arthroplasty and more particularly to devices, methods and techniques which can be used by a surgeon to achieve appropriate placement of a femoral component.

### BACKGROUND

Among the many factors which can lead to a positive outcome (e.g., an optimal outcome) in knee arthroplasty, sizing and positioning of the components are among the most significant and determinative, or even critical. This is particularly true with the femoral component, which needs to be correctly or otherwise appropriately placed in the three translational axes and the three rotational axes. Of these axes, one that plays a key role in the patient's outcome is the size and translation in the anterior-posterior (AP) direction. If a component is too large or placed too far anteriorly, it can lead to “overstuffing” the patellofemoral compartment, which can lead to patella issues and pain. If the anterior cut for a component is too posterior, a deep resection can be made in the anterior cortex of the femur, weakening it and potentially contributing to a fracture. The posterior position is also important. If the femoral component is too large and/or placed too far posteriorly, the patient has potential to lose flexion, whereas the opposite can result in flexion instability. Various techniques are used by surgeons to position the femoral component, each of which involve various compromises. Some surgeons choose to primarily use “measured resection” techniques, where specific amounts of bone are removed, with subsequent soft tissue releases to fine-tune the ligaments. Other surgeons use “gap balancing” techniques, where various cuts are specifically planned to balance the soft tissues around the knee in both flexion and extension.

With all of these known techniques, it is still critical to choose a correct size for the femoral component, and make the required cuts in the appropriate position with regards to the AP translation axis (AP position). For some femurs, the anatomic size of the femur lines up exactly with one of the specific sizes in the particular system used by a surgeon. For many patients, however, the femur is in-between sizes, and a surgeon must compromise in one direction or another. In a posterior-referencing system, the focus is on matching the distal and posterior cuts to balance the ligaments optimally, accepting a mismatch anteriorly. This has the potential to cause “notching” or “overstuffing”. Other systems are anterior-referencing, where the focus is on making the anterior cut in the optimal position, accepting a potential mismatch on the posterior cut. Even when using gap-balancing techniques, the focus is primarily on the level of the posterior cut with a potential mismatch on the anterior cut.

Most current sets of instrumentation are designed to be anterior-referencing, posterior-referencing or gap-balancing. There are a few companies that have included positioning holes for both anterior and posterior in the same instrument, but in practical use, a surgeon may only use it in either an anterior-referencing OR posterior-referencing technique as one set of positioning holes is associated with a fixed distance from an anterior landmark and another set might be associated with a fixed distance from a posterior landmark, which posterior landmark may be either on the femur or on a part of the tibia.

In addition to forcing a surgeon to choose a specific approach to positioning (anterior/posterior/gap), there are some practical limitations to current instrumentation sets. Because of size and/or space constraints, some manufacturers have chosen to only support one approach for a given product (i.e., only posterior referencing). For these manufacturers, this makes it such that a surgeon who prefers anterior referencing cannot use their system. Other manufacturers have made 2 independent sets of instruments, with one for anterior referencing and another for posterior referencing. In addition to the expense of manufacturing and supporting these two sets of instruments, there are other disadvantages. Representatives often have to assure or otherwise provide access to most instrument sets, such that an “anterior-referencing surgeon” gets the appropriate set at their facility on the appropriate day, and vice versa.

This switching of instrumentation according to location or surgeon preference also has potential for problems during a case. If a sizer from an anterior-referencing system is accidentally placed in a set of instruments with posterior-referencing blocks, and if this isn't recognized by the surgeon at the time of the procedure, a poor outcome can result. Even if the surgeon does recognize the mismatch, it can often require unnecessary time for a patient receiving anesthesia to find and sterilize the matching instrument sets.

### SUMMARY

The present disclosure is directed to devices, methods and techniques that can be used by a surgeon to reference both anterior and posterior landmarks simultaneously to find an appropriate (e.g., optimal) hybrid reference point to minimize error in both the anterior and posterior directions. The disclosure is also directed to devices, methods and techniques that can be used by manufactures to develop a single hybrid set of instruments to allow for anterior and posterior referencing, as well as measured-resection and gap-balancing surgeon preferences.

Additional aspects and advantages will be apparent from the following detailed description of preferred embodiments, which proceeds with reference to the accompanying drawings.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates graphs showing how the normal anatomic variation in femur size relates to component sizing in knee arthroplasty.

FIG. 2 shows lateral views of several examples of a distal femur and referencing techniques at the same.

FIG. 3 illustrates several methods from calculus that are used in knee arthroplasty to help minimize the effect of mismatch.

FIG. 4 is a lateral view of a femur to demonstrate various uses of various landmarks.

FIG. 5 includes several graphs that demonstrate the various types of mismatch using different referencing techniques.

FIG. 6 is a graph representing how mismatch is addressed by embodiments in accordance with the present disclosure.

FIG. 7 is a perspective view of a hybrid referencing device, according to one embodiment of the present disclosure.

FIG. 8 shows a diagrammatic view of a knee and some examples of posterior landmarks of the knee that are used in knee arthroplasty.

FIG. 9 is a view of the back of a hybrid referencing device, according to one embodiment of the present disclosure.

FIG. 10 is a view of the back of a hybrid referencing device, according to one embodiment of the present disclosure.

FIG. 11 illustrates a representation of how different frames of reference can be used conceptually to implement concepts from the present disclosure in different embodiments.

FIG. 12 depicts a hybrid referencing device, according to an embodiment of the present disclosure.

FIG. 13 is hybrid referencing device 1300, according to another embodiment of the present disclosure.

### DETAILED DESCRIPTION

The present disclosure is directed to devices used in performing a knee arthroplasty, and more particularly to devices, methods and techniques that can assist in sizing and/or positioning a femoral component in an arthroplasty. Example embodiments are provided, including with reference to the accompanying drawings so this disclosure can convey the scope to those who are skilled in the art. Numerous specific details are provided as examples of specific components, devices and methods, to provide a thorough understanding of the embodiments in the present disclosure. These specific details need not be employed and example embodiments may be embodied in many different forms, and neither should be construed to limit the scope of the disclosure. In some example embodiments, well-known processes, well-known structures and well-known technologies are not described.

When performing a knee arthroplasty, it is critical to position the various final components correctly in order to obtain a positive (e.g., an optimal) clinical outcome. Each component in a knee arthroplasty, including the femoral component, has six-degrees of freedom, including translation in three orthogonal axes as well as rotation in each of the three axes. This also includes any combination of these degrees of freedom. As is well-known to a person of ordinary skill, in a standard procedure to prepare the femur for a final component, the distal femoral cut is often made first. Important next steps include, but are not limited to: measuring the distal femur in the anterior-posterior to determine the most appropriate size component and determining the correct position of the component, including translation in the anterior-posterior direction and internal-external rotation.

In an anterior-referencing system, a landmark related to the anterior part of the femur is defined to determine a level of an ideal anterior cut. This anterior landmark is generally on the anterior cortex of the femur. When using an anterior-referencing system, the planned anterior cut is based on this anterior landmark and a planned saw cut level is defined to meet this landmark. Most instrumentation systems currently use a sizing and positioning device, which allow one or more methods for making guide marks on the femur. These guide marks could be holes in the bone, pins, or any other method. These guide marks are then generally used to align a separate cutting block which has saw guides used to make the actual cuts. Alternatively, one or more saw guides can be built into the femoral sizing and positioning device. The planned posterior cut is a fixed distance from the planned anterior cut at a distance corresponding to the planned anterior-posterior size of the femoral component. Hence, both the planned anterior and posterior cuts are defined at a fixed translational distance from the anterior landmark. When using an anterior-referencing system, the distance from a posterior landmark on the femur and/or the proximal tibia will vary. This has the potential to introduce error when balancing flexion and extension gap.

In a posterior-referencing system, a landmark related to the posterior part of the femur and/or the proximal tibia is defined. The posterior condyle of the femur is often used as a posterior landmark in a measured-resection technique, while the tibia is often used as a posterior landmark in a gap-balancing technique. The level of the ideal posterior cut is therefore defined in relation to this posterior landmark, with a planned posterior cut to be made at this ideal posterior level. The planned anterior cut is set at a fixed distance from the planned posterior cut at a distance corresponding to the planned anterior-posterior size of the femoral component. When using a posterior-referencing system, the distance of the planned anterior cut may vary from the ideal anterior cut. If it does correspond exactly with the ideal anterior cut, the planned anterior cut will line up with the anterior landmark, but otherwise there will either be too much bone removed anteriorly (leading to notching) or too little removed (leading to overstuffing).

While many manufacturers of knee arthroplasty systems have introduced more sizes in order to reduce the gaps between sizes, and hence reduce error caused by mismatch between a patient's anatomy and a component available in a given system, there is a practical limit to the amount of instruments and inventory to accommodate all these sizes. Because the anatomy of most patients doesn't correspond exactly to one of the sizes, there will be error introduced. In an anterior-referencing system, all of the potential mismatch will involve the posterior cut. In a posterior-referencing system, all of the potential mismatch will involve the anterior cut. In both cases, the maximum mismatch will be when a femur is in the middle of two sizes, as when a femur gets closer to a specific size, the surgeon generally chooses that size.

The present disclosure is directed to devices, methods and techniques to enable a “hybrid-referencing” system which uniquely references both an anterior landmark and a posterior landmark to define a reference position. By referencing both anterior and posterior landmarks, the planned position of the anterior and posterior femoral cuts can be determined to “split the difference” of any mismatch, thereby minimizing variation from the ideal anterior and posterior cuts.

For example, many current knee arthroplasty systems have a 3 mm gap between sizes (i.e., between size 3 and 4). If a femur is exactly a size 3 or a size 4, that size can be used and there isn't any mismatch. But for a femur that is directly between two sizes (e.g., size 3.5), there will be up to a 1.5 mm mismatch. In an anterior-referencing system, this entire 1.5 mm mismatch will either make the posterior part of the component too small, if a size 3 is chosen, or too large, if a size 4 is chosen. In a posterior-referencing system, this entire 1.5 mm mismatch would be anterior, either leading to notching the femur if a size 3 is chosen, or overstuffing the patella if a size 4 is chosen.

In utilizing a hybrid-referencing system such as described in this disclosure, the mismatch will be distributed more evenly. For this example, it would create a 0.75 mm mismatch anteriorly and a 0.75 mm mismatch posteriorly. As a frame of reference, this is less than the width of most sawblades used in arthroplasty and within the margin of error for making the planned resections.

In addition to allowing for a surgeon to more predictably use an appropriate (e.g., an optimal) position for the femoral component, a hybrid sizing and positioning device has the added benefit of allowing for a single instrument system to be used for all surgeons, rather than requiring separate anterior-referencing and posterior-referencing systems to support different surgeons'techniques.

Additionally, utilizing a hybrid-referencing system described in this disclosure can support times during a case when a surgeon wants to change referencing systems. For example, in a patient with osteoporotic bone where the surgeon absolutely does not want to notch the femur, a method will be shown where the surgeon can follow the anterior cortex at the expense of potential flexion-extension mismatch. Conversely, in a high-demand patient with strong bone, the surgeon might choose to have no mismatch posteriorly and all of the mismatch anteriorly. All of these are possible and can be done during the procedure using the hybrid-referencing system described in this disclosure without needing to open any additional instrumentation.

Examples of embodiments of the present disclosure are described below with reference to the drawings and in a context of knee arthroplasty. An instrument system which includes devices, techniques and methods to optimally size and position a femoral component in a knee arthroplasty can be enhanced by embodiments of the present disclosure.

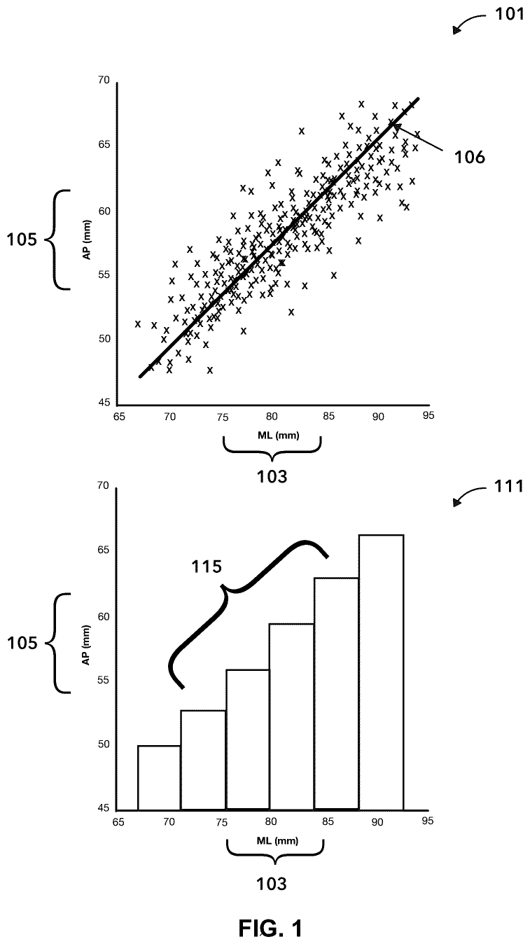

FIG. 1 illustrates graphs showing how the normal anatomic variation in femur size relates to component sizing in knee arthroplasty and leads to a need for devices, techniques and methods described in the present disclosure. Graph 101 shows a sample of distal femur sizes in a wide range of people. The ML (medial-lateral) dimension 103 (e.g., a width) of the femur is plotted on the x-axis and the corresponding AP (anterior-posterior) dimension 105 of the femur is plotted on the y-axis. As can be seen, there is a roughly linear correspondence between the ML dimension 103 and the AP dimension 105. Designers of knee arthroplasty systems use graphs like this to define ratios between the ML and AP dimensions of their various sizes. While there is a general line of linear regression as plotted, as can be seen, there is significant individual variation. Additionally, when enough points are plotted, it is evident that sizes of the distal femur in the AP dimension 105 are continually variable.

The bar graph 111 illustrates an example of AP dimensions 105 and corresponding sizes 115 of femoral components used in knee arthroplasty. Designers of the different systems choose a number of sizes 115 to support and define AP dimensions 105 and ML dimensions 103 corresponding to those sizes 115. Choosing the possible sizes is a balance between having too few sizes, which can lead to large gaps between sizes, and having too many sizes, as each size requires specific instrumentation and inventory, which can add to the expense of the system. Importantly, while femoral components only come in discrete sizes 115, normal human anatomy follows a more or less continuous range 106.

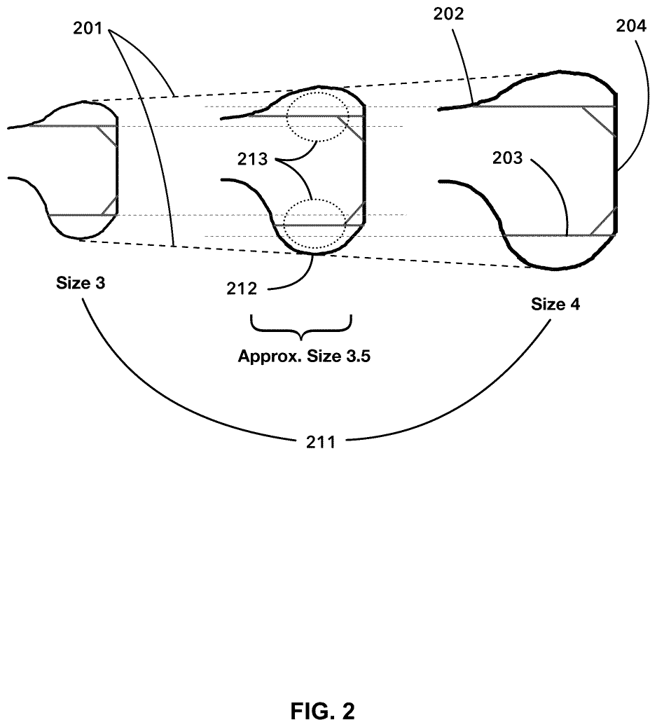

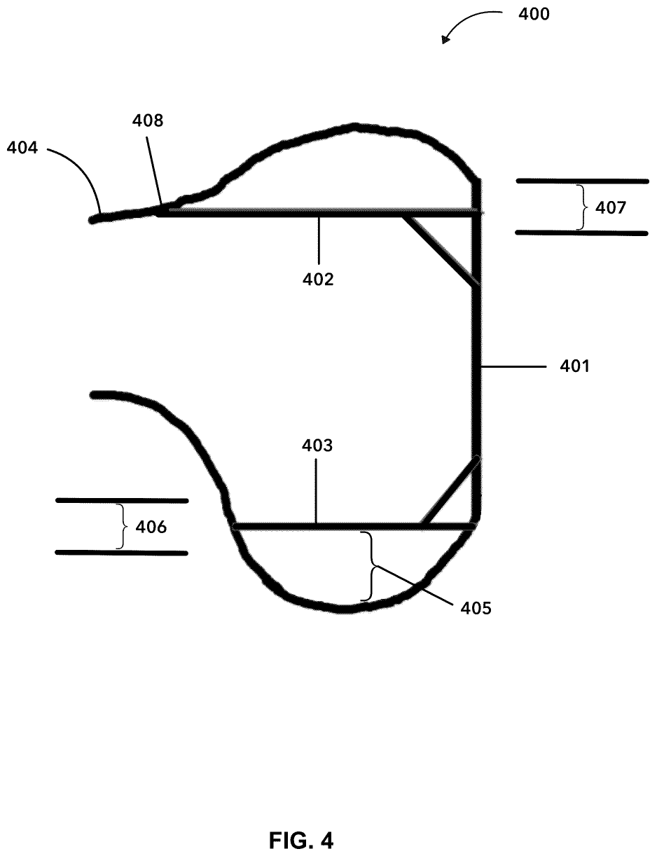

FIG. 2 shows lateral views of several examples of a femur. FIG. 2 also demonstrates a potential for mismatch between continuously variable patient anatomy possibilities and the discrete sizes 211 of components available for knee arthroplasty. Two broken lines 201 show the continuously variable patient anatomy possibilities in the AP dimension between a femur corresponding to size 3 and size 4. A more detailed description of the locations for the ideal anterior cut and the ideal posterior cut is given in FIG. 4, but this FIG. 2 shows the potential mismatch when trying to match a continuous variable (anatomic size) with a discrete variable (i.e., available component size). For instances when a patient's anatomy corresponds to an available size, an ideal position for the femoral component can be made. Ideally, the anterior cut 202 lines up with the anterior cortex of the femur. Ideally, the posterior cut 203 is designed to remove an equivalent amount of bone as previously removed on with the distal cut 204 to balance the flexion-extension gap and optimize functionality.

For many femurs, however, the measured AP dimension of the femur falls between two available sizes—e.g., in the illustrated situation the equivalent of a 3.5 size 212. The surgeon can decide if they want to cut the femur that is generally equivalent to size 3.5 to match a size 3 component, in which case it is undersized. Alternatively, the surgeon can decide to cut it to match a size 4, in which case it is oversized. In making either choice, there is a potential mismatch 213 represented in the space between the dotted lines representing an available component size and the solid lines representing the ideal location for the anterior or posterior cut. The potential mismatch 213 needs to be addressed-ideally in a way that least compromises the patient outcome.

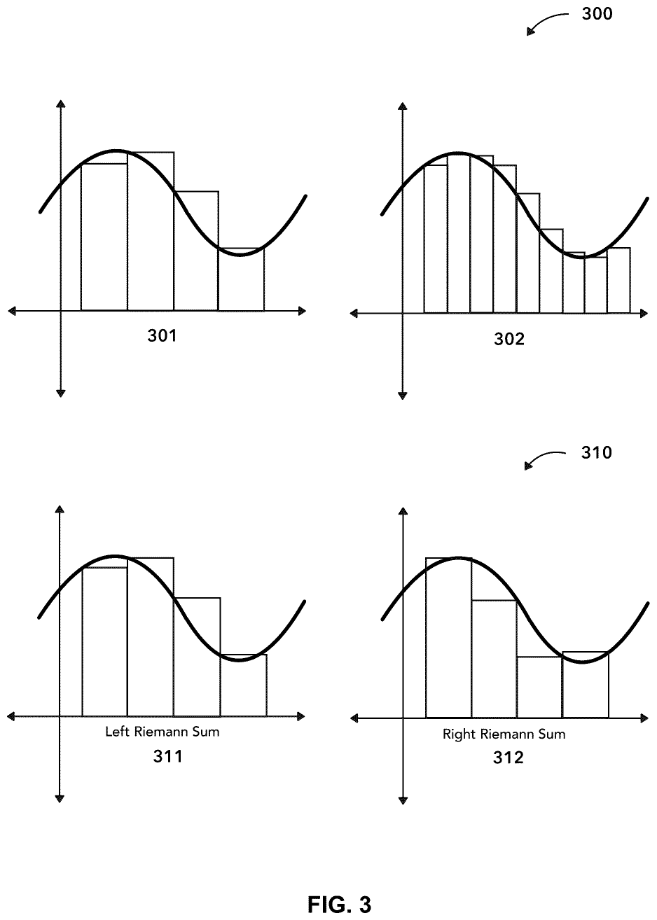

FIG. 3 illustrates several methods from calculus that are used in knee arthroplasty to help minimize the effect of mismatch. From calculus, the first set of graphs 300 show one method of minimizing error when trying to match discrete and continuous functions. As seen in the left graph 301, when the bars are wide, there is potential for more significant error as seen in the areas where the bars extend past or don't quite meet the continuous function. As seen in the right graph 302, the bars can more closely match the continuous function by making them more narrow. While calculus takes this to the limit by letting the width of the bar approach zero in integrals, this is not possible practically in arthroplasty and component sizing as a subset of discrete sizes must practically be chosen.

However, the concept of more narrow bars is reflected in the practice of making more sizes. The overall range of sizes from smallest to largest is generally similar between manufacturers, as sizes need to be available from the smallest to the largest person. Between the minimum and maximum, however, some manufacturers may have 5-6 sizes between these extremes while other manufacturers may have 9-10 sizes. While having more sizes lets a surgeon more closely match an individual patient's anatomy, there are tradeoffs as more sizes require more instrumentation, more inventory, and more expense.

Once the sizes for any given system are determined, there is always going to be a potential mismatch when a particular patient's anatomy falls between available sizes. There are different techniques generally used to manage or otherwise address a mismatch. Again, the fundamental concept comes from calculus. In the second set of graphs 310, the same function with the same width of bars as in 301 is shown. The difference between these two graphs 311, 312 is that when using a left Reimann sum (see graph 311), the top left corner of the bar is designed to meet the continuous function, while when using a right Reimann sum (see graph 312), the top right corner of the bar is designed to meet the continuous function. Depending on various factors, including the details of a specific function, sometimes a left Reimann sum (see graph 311) gives a more accurate solution, while other times a right Reimann sum (see graph 312) is more accurate.

As will be shown in FIG. 4, a similar concept is used in knee arthroplasty. For patients where the AP dimension of the femur doesn't match an available component size exactly, different referencing points can be used, with pros and cons to each one. These referencing points or approaches can include anterior-referencing, where a landmark related to the planned anterior cut of the femur is used, and posterior-referencing, where a landmark related to the planned posterior cut of the femur is used. As will be described below, these landmarks can be on the distal end of the femur or on the proximal end of the tibia.

FIG. 4 is a lateral view of a femur 400 to demonstrate various uses of various landmarks. Advantages and disadvantages of each of them are discussed. When a surgeon performs a knee arthroplasty, the distal cut 401 on the femur 400 is often made first. Other instrumentation outside the discussion and scope of the present disclosure is used to make this cut at a surgeon's preferred angle and depth of resection. This distal cut 401 is often planned to resect 9 to 10 mm of bone from the most distal portion of the femur, although it can vary. The two main additional cuts are the anterior cut 402 and the posterior cut 403. The distance between these cuts is fixed once a size of a component is chosen by a surgeon and is designed to match the AP dimension of the component that will be implanted.

For the anterior cut 402, it is generally accepted that an ideal location matches the anterior cortex 404 of the femur 400. The anterior cortex of the femur is the smooth, convex front surface of the femur shaft, often use in orthopedics as a reference landmark, such as for aligning knee implants. For the posterior cut 403, a generally accepted goal is to resect an amount of bone 405 from the posterior condyles of the femur such that, when compared to the amount of bone resected with the distal cut 401, the knee is well-balanced in flexion and extension. The posterior condyles of the femur are the two large, rounded, smooth, and convex bony projections on the rear, bottom (distal) end of the femur.

In a patient whose native anatomy matches a component size available in a given system, choosing an AP translational position of the planned cuts is fairly straightforward. The planned anterior cut 402 can be chosen to match the anterior cortex 404 of the femur 400. The planned posterior cut 403 can be chosen such that the amount of resected bone 405 allows the knee to balance in flexion and extension. A more challenging situation arises when the patient's anatomy does not correspond to a specific component size of a given system, especially when the anatomy is somewhat equal between two sizes. In this case, just like with the Reimann sums, there are advantages and disadvantages to the options currently available.

In an anterior-referencing system, the primary positioning landmark is generally the anterior cortex 404 of the femur 401. The position of the cuts is chosen such that the planned anterior cut 402 matches the chosen landmark, and any mismatch between the patient's anatomy and the chosen size results in variability 406 in the position of the posterior cut 403. As previously described, this can lead to problems with flexion-extension imbalance, particularly as the amount of mismatch increases.

In a posterior-referencing system, the primary posterior landmark is generally one or both posterior condyles of the femur. The position of the cuts is chosen such that the planned posterior cut 403 matches a known displacement from the chosen landmark, and any mismatch between the patient's anatomy and the chosen size results in variability 407 in the anterior cut 402. As previously described, this can lead to problems with notching and potentially weakening the femur if a smaller size creates a notch 408. It can also lead to problems with overstuffing the patellofemoral joint if too little bone is removed.

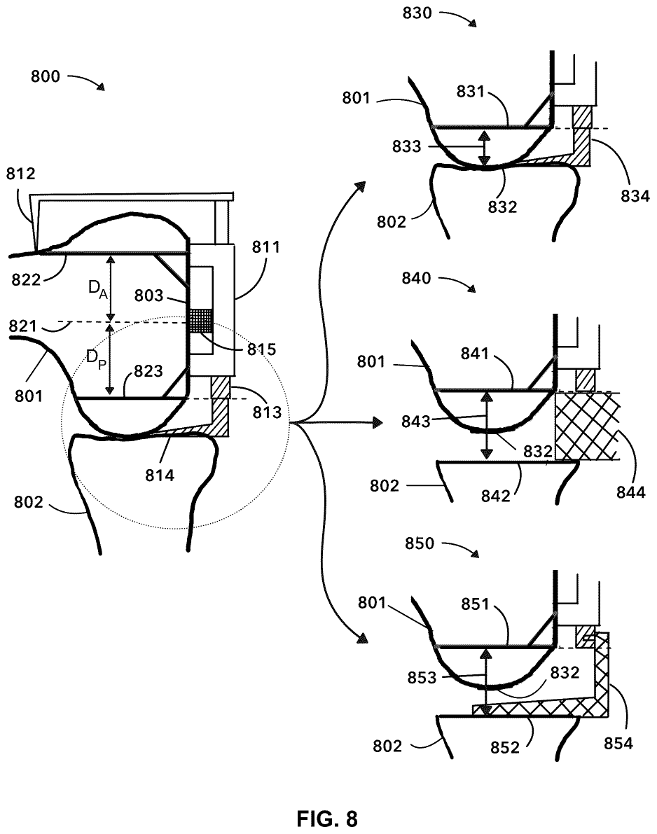

In a gap-balancing system, the primary focus is also the position of the posterior cut 403. The main difference between a gap-balancing system and a posterior-referencing system with regards to positioning of the cuts is the choice of the primary positioning landmark. Instead of using a landmark on the femur, in gap balancing the landmark is generally on the tibia. This difference is explained more thoroughly with reference to FIG. 8. Because of the focus on the position of the posterior cut 403 and how it works with the distal cut 401 to emphasize flexion-extension balance, gap-balancing systems have the same potential problems as posterior-referencing system, as all potential mismatch is introduced anteriorly 407.

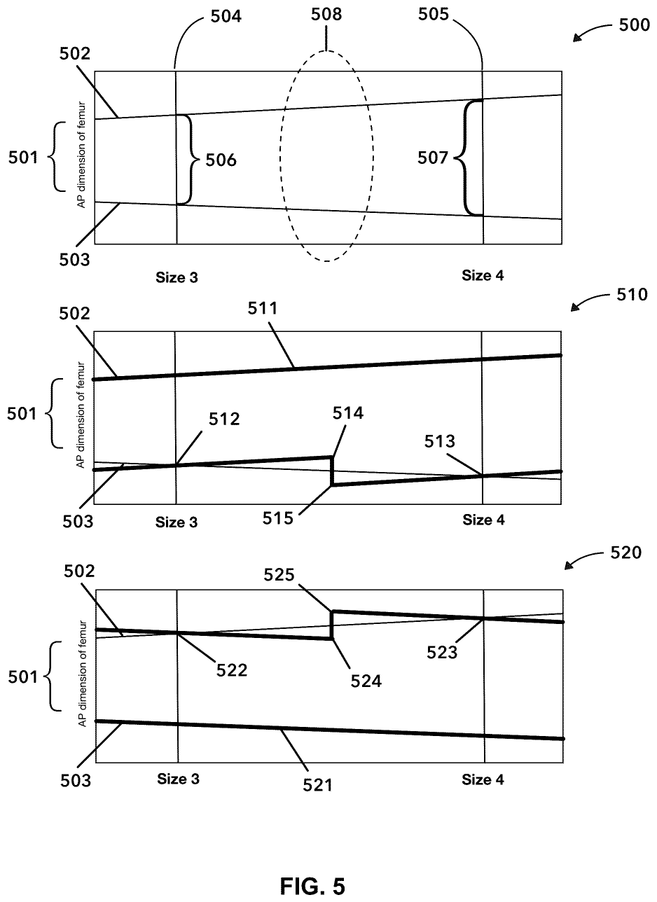

FIG. 5 includes several graphs 500, 510, 520 that demonstrate the various types of mismatch using different referencing techniques, along with the potential downsides of each one. Graph 500 is a representation of the continuous sizes found in natural femurs as encountered in knee arthroplasty. The y-axis 501 represents a position of an ideal anterior cut 502 and an ideal posterior cut 503 on a femur based on the respective landmarks described in FIG. 4. The left side of the graph, when the position of the ideal anterior cut 502 and ideal posterior cuts 503 are closer together represents smaller femurs, with the distance between the ideal anterior cut 502 and ideal posterior cut 503 increasing towards the right of the graph, representing larger femurs. The linear nature of these lines 502, 503 represents the continuous nature of possibilities.

For some patients, the ideal distance in the AP dimension matches an available component size in a given system. In this graph, a size 3 504 and a size 4 505 are represented. For the size 3 504, the AP distance of the size 3 component 506 matches the AP distance between the ideal anterior cut 502 and the ideal posterior cut 503. Similarly, for the size 4 505, the AP distance of the size 4 component 507 matches the AP distance between the ideal anterior cut 502 and the ideal posterior cut 503. In these cases, there is no mismatch between the ideal cuts 502 (and the patient's anatomy) and the component size. The potential issues arise for femurs whose ideal cuts are between sizes 508. In this area, the AP distance between the ideal anterior cut 502 and the ideal posterior cut 503 does not match the AP distance of the available size 3 component 506 or the available size 4 component 507. A surgeon must decide whether to use a size that is smaller or bigger, and where to position the component to deal with the mismatch.

Graph 510 is a representation of how the mismatch is handled in an anterior-referencing system. In this graph 510, the same lines representing the ideal anterior cut 502 and the ideal posterior cut 503 are shown. In an anterior-referencing system, the representation of the planned anterior cut 511 based on a fixed relation to an anterior landmark is shown by a heavier line. The planned anterior cut 511 is based on the ideal. As seen by the solid line representing the planned anterior cut 511, there is no mismatch anteriorly through the range of possible dimensions for the patient's femur as the planned anterior cut 511 already lines up with the ideal anterior cut 502.

Posteriorly, when the patient's femur matches a size 3 in the AP dimension 512 or a size 4 in the AP dimension 513, there is also no mismatch posteriorly. However, between available component sizes, approximately a size 3.5, the surgeon must decide which size to use. If a size 3 is chosen, the resulting posterior cut 514 will resect more posterior femur than the ideal posterior cut location 503 and an increased flexion gap can occur, leading to a flexion-extension imbalance. If a size 4 is chosen, the resulting posterior cut 515 will resect less posterior femur than the ideal posterior cut location 503 and a decreased flexion gap can occur, again leading to a flexion-extension imbalance. This flexion-extension imbalance between sizes can be enhanced as all potential mismatch between the AP dimension of an available component and the AP dimension of the ideal cuts based on a patient's anatomy is addressed posteriorly in an anterior-referencing system.

Graph 520 is a representation of how the mismatch is handled in a posterior-referencing system. In this graph 520, the same lines representing the ideal anterior cut 502 and the ideal posterior cut 503 are shown. In a posterior-referencing system, the posterior cut is based on a fixed relation to a posterior landmark with the emphasis on the flexion-extension balance. The planned posterior cut 521 is based on the ideal. As seen by the solid line representing the planned posterior cut 521, there is no mismatch posteriorly through the range of possible dimensions for the patient's femur as the planned posterior cut 521 already aligns with the ideal posterior cut 503. When the patient's femur matches a size 3 in the AP dimension 522 or a size 4 523, there is also no mismatch anteriorly.

Between available component sizes—approximately a size 3.5—a surgeon must decide which size to use. If a size 3 is chosen, the resulting anterior cut 524 will resect more anterior bone than the ideal anterior cut location 502, leading to potential femoral notching with resulting weakening of the femur. If a size 4 is chosen, the resulting anterior cut 525 will resect less anterior femur than the ideal anterior cut location 502, potentially leading to overstuffing of the patellofemoral joint. Patellofemoral overstuffing is a post-total knee arthroplasty complication where the combined thickness of the femoral and patellar prosthetic components exceeds the original bone and cartilage, increasing anterior pressure. It is characterized by an increased patellofemoral distance, often resulting in stiffness, anterior knee pain, and decreased range of motion. These anterior issues between sizes stem from the concept that all of the potential mismatch between the AP dimension of an available component and the AP dimension of the ideal cuts based on a patient's anatomy is addressed anteriorly in a posterior-referencing system.

In both anterior-referencing and posterior-referencing systems currently available, there are methods where a surgeon can manually modify the position of the cuts. These may include, but are not limited to, one or more of: having additional marking holes at a different offset from the primary holes, having a dial or other method where a surgeon can manually move the positioning guide to a different position, and having offset capabilities on a different instrument. All of these calculations must be done by a surgeon and performed manually.

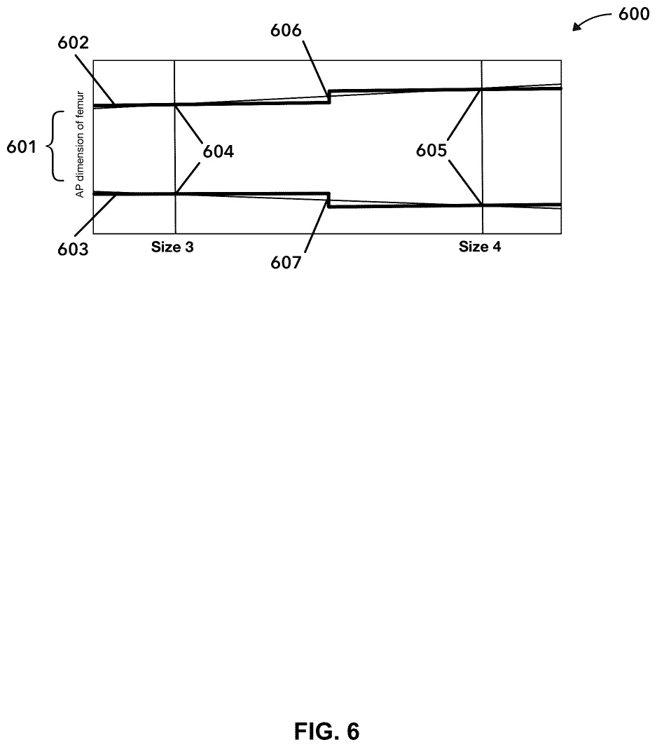

FIG. 6 is a graph 600 representing how mismatch is addressed by embodiments in accordance with the present disclosure. As in FIG. 5, the y-axis 601 represents a position of an anterior cut and a posterior cut on a femur based on the respective landmarks and lines representing the locations of an ideal anterior cut 602 and an ideal posterior cut 603 are shown, with smaller femurs represented toward the left and larger femurs represented toward the right. As in both anterior-referencing and posterior-referencing, when the AP distance between the ideal anterior and posterior cuts 602 603 on patient's femur matches the AP distance of a size 3 604 or a size 4 605 component, the anterior and posterior cuts match the optimal location as in graph 510 and graph 520 of FIG. 5. The present disclosure describes devices, techniques and methods for referencing both anterior and posterior landmarks together to identify a location for the planned cuts in the femur that splits any potential mismatch between both the anterior cut and the posterior cut, minimizing the absolute amount of mismatch in either cut. The graph 600 of FIG. 6 illustrates effects of referencing both anterior and posterior landmarks together to divide the potential mismatch substantially evenly between the anterior cut and the posterior cut.

Using the same example approximating a size 3.5, the resulting mismatch with a hybrid-positioning system as described in this disclosure can be seen graphically in the graph 600 of FIG. 6. On the anterior cut 606, there can still be a potential mismatch with the same issues described above with reference to FIG. 5, but the amount and clinical significance can be reduced. It is the same with the posterior cut 607, where the potential mismatch is also reduced. Mathematically, when a patient's femur matches an available size exactly, such as a size 3 604 or a size 4 604, there is 0 mm mismatch. In an extreme or “worst-case” situation, where the patient's femur is exactly between two sizes, the total mismatch (e.g., the distance between an ideal anterior cut 602 and an ideal posterior cut 603) is approximately equal to half the gap between sizes. For many modern instrument sets with 3-4 mm gaps between sizes, this total mismatch midway between sizes would be 1.5-2 mm. In an anterior-referencing system or a posterior-referencing system, all of this mismatch would affect the posterior or anterior cut respectively. In a hybrid-referencing system, because the total mismatch is split between the anterior cut 606 and the posterior cut 607, either cut would individually have a maximal 0.75-1 mm mismatch. This is less than the typical width of a saw blade used to make the cuts, so the potential clinical impact is reduced compared with a system or approach where the entire mismatch ends up in only the anterior cut (e.g., anterior cut 524, 525 of FIG. 5) or the posterior cut (e.g., posterior cut 514, 515 of FIG. 5).

In the present disclosure, by referencing one or more anterior landmark and one or more posterior landmark, an instrument can be designed that optimally performs any adjustment to anterior-posterior translation that a surgeon may choose to manually do with existing systems.

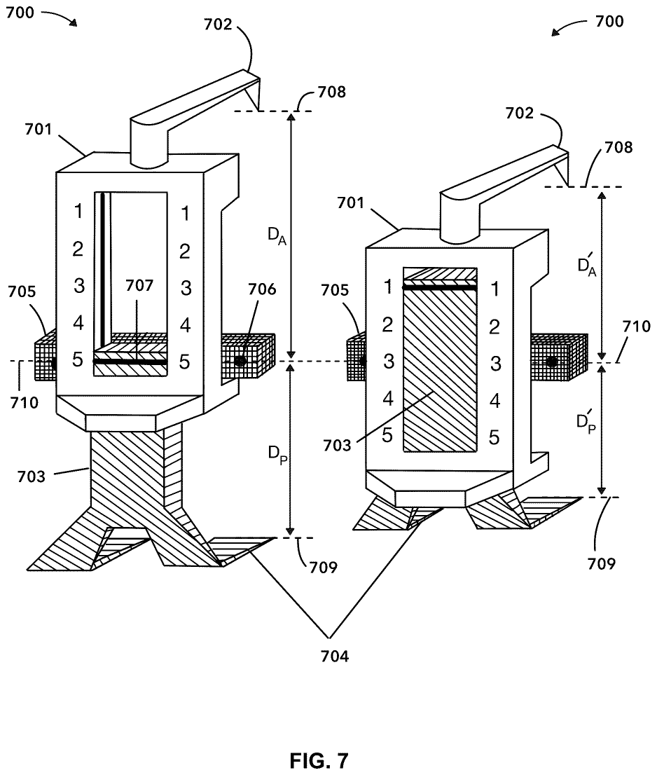

FIG. 7 is a view of a hybrid referencing device 700, according to one embodiment of the present disclosure. The hybrid referencing device 700 is depicted in two different measuring states or configurations corresponding to measuring anatomy (i.e., femurs) of different sizes. The hybrid referencing device 700 can be a sizing and/or positioning device for determining a size and/or a position for a femoral component in knee arthroplasty. The hybrid referencing device 700 can measure, obtain or otherwise detect the AP distance of a distal femur, including for various sizes or dimensions of femurs along a full continuum. While the referencing device 700 can measure multiple sizes of a distal femur, for the sake of this example, two different positions (or measuring states) are shown corresponding to a larger size (on the left) and a smaller size (on the right). These different positions will collectively be described as the referencing device 700, and corresponding features of both positions will be referenced similarly.

The referencing device 700 can include an anterior referencing component 701, a posterior referencing component 703, and a positioning component 705. The anterior referencing component 701 and posterior referencing component 703 can be used to measure an anterior-posterior (AP) distance (also referred to as an anteroposterior distance) related to a distal femur in order to determine a corresponding size femoral component of a knee arthroplasty system. The positioning component 705 can be used to determine one or more of: translation and rotation, in order to assist in positioning a corresponding femoral component from an arthroplasty system. The positioning component 705 can include a feature 706 (e.g., a drill hole, a pin hole, a punch guide, a saw guide, a notch and a defined surface) to enable marking one or more locations on a distal femur. A mark 707 can be present on the one or more components indicating or corresponding to an available size component for a measured anterior-posterior distance on a femur.

The anterior referencing component 701 can include an anterior referencing stylus 702 in any shape necessary to define, engage, or otherwise obtain a position of one or more points 708 associated with an anterior portion of a distal femur. The one or more points 708 can be one or more landmarks related to an anterior part of a distal femur. For example, the anterior cortex can be a landmark engaged by the anterior referencing stylus 702. The posterior referencing component 703 can include a posterior referencing stylus 704 in any shape (e.g. tabs, feet, etc.) appropriate to define, engage, or otherwise obtain a position of one or more points 709 associated with a posterior part of the distal femur. The one or more points 709 associated with the posterior part of the distal femur can be one or more landmarks related to a posterior part of the distal femur. For example, the posterior condyles can be landmarks engaged by the posterior referencing stylus 704. In some embodiments, the posterior referencing stylus 704 can include one or more flat extensions which can be placed against one or more posterior condyles of a femur (e.g. posterior feet). Alternatively, the posterior referencing component 703 can be designed to be used in a gap-balancing technique where the posterior landmark is located on the tibia.

The positioning component 705 can move in a way related to both the anterior referencing component 701 and the posterior referencing component 703, while being a variable distance from both the anterior referencing component 701 and the posterior referencing component 703. The positioning component 705 can therefore be located a variable distance, marked D_{A}, from one or more anterior landmarks 708, as associated with an anterior referencing stylus 702. It can simultaneously be located a variable distance, marked D_{P}, from a posterior landmark 709, which can include one or more of: a posterior portion of a distal femur, multiple posterior portions of a distal femur, and a position on a proximal tibia, as associated with a posterior referencing stylus 704.

The variable distance to both the anterior and posterior referencing points is demonstrated. In the measuring state of the device 700 corresponding to a larger size (on the left of FIG. 7), the positioning component 705 is located near the bottom of the anterior referencing component 701 and the top of the posterior referencing component 703. In the measuring state corresponding to a smaller size (on the right of FIG. 7), the positioning component 705 is located more towards the middle of both the anterior referencing component 701 and the posterior referencing component 703. Stated in a different manner, the distance from the positioning component 705 to both the anterior referencing stylus 702 and the posterior referencing stylus 704 for the measurement state or configuration corresponding to a larger size is larger than the corresponding distances for the measurement state of configuration corresponding to a smaller size.

Another demonstration of how the positioning component 705 can move in a way related to both the anterior referencing component 701 and the posterior referencing component 703, while being a variable distance from both the anterior referencing component 701 and the posterior referencing component 703, is shown. In both the configuration corresponding to the larger size and the configuration corresponding to the smaller size, an anterior reference landmark 708 and a posterior reference landmark 709 are identified. The distance between the anterior landmark(s) 708 and posterior landmark(s) 709 and a reference location 710 are shown. In both configurations, distance D_{A} represents a distance between an anterior reference landmark 708 and a reference location 710, while distance D_{P} represents a distance between a posterior reference landmark 709 and a reference location 710. Although in both depicted configurations the distances D_{A} and D_{P} are related to the distance between the anterior reference landmark 708 and the posterior reference landmark 709, it can be seen that distance D_{A} is not equal to distance D′_{A} and distance D_{P} is not equal to distance D′_{P}. The distance D_{A} and the distance D_{P} are each larger for the larger measuring state (on the left) than distance D′_{A} and distance D′_{P}, respectively, for the smaller measuring state (on the right).

FIG. 7 therefore shows that the distance from the positioning component 705 to both the anterior referencing stylus 702 and the posterior referencing stylus 704 is variable. This contrasts with existing anterior-referencing systems which fix the distance to an anterior stylus with a variable distance to a posterior stylus, in which D_{A} in a measurement of a larger size would equal D′_{A} in a measurement of a smaller size. It also contrasts with existing posterior-referencing systems which fix the distance to a posterior stylus with a variable distance to an anterior stylus, in which D_{P} in a measurement of a larger size would equal D′_{P} in a measurement of a smaller size. Although, in some currently available systems, a reference location 710 can be fine-tuned manually (e.g., by a surgeon), still the location 710 is based from and fixed relative to either an anterior reference point 708 or a posterior reference point 709.

Once an appropriate position is marked on the distal femur using the referencing device 700, a universal set of femoral cutting blocks corresponding to the various femoral component sizes can be used by surgeons using the hybrid-referencing system described in the present disclosure, as well as by one or more of: anterior-referencing and posterior-referencing surgeons. This eliminates the potential need for having multiple sets of cutting blocks which have similar cutting slots but different positions for locating the cutting block related to the one or more sizing and positioning components.

FIG. 8 shows a diagrammatic view 800 of a knee and some examples of posterior landmarks of the knee that are used in knee arthroplasty. The view 800 is a lateral view of a knee with a sizing and positioning device in place. A femur 801 and a tibia 802 are shown at approximately 90 degrees of flexion. In this diagrammatic view 800, a distal femoral cut 803 has already been made using a standard technique beyond the scope of this disclosure. An anterior component 811 of the sizing and positioning device is shown resting against the cut distal femoral cut 803 surface with an associated anterior stylus 812 resting against the anterior cortex of the femur. The posterior component 813 of the sizing and positioning device is also shown, with an associated posterior stylus 814 resting against the posterior condyles of the femur 801. A reference component 815 is shown resting against the distal femoral cut 803 surface of the femur 801. The reference component 815 is used to define a reference location 821. In combination with a known component size as measured by the sizing and positioning component and reference location 821, a distance D_{A} to a planned anterior cut 822 and a distance D_{P} to a planned posterior cut 823 can be defined. These distances are built into an appropriate cutting block corresponding to a given component size. The area in this view 800 enclosed in the dotted circle are expanded in three example views 830, 840, 850 showing different examples of posterior landmarks.

The first view 830 shows a representation of a typical posterior-referencing system using a measured resection technique. The planned posterior cut 831 is chosen such that the amount of bone that will be removed from the posterior femoral condyle 833 is equivalent in thickness to the bone previously removed from the distal femur. By removing the same amount of bone, an ideal flexion-extension balance can result. To measure the amount of bone resected, there is typically a thin “feeler” 834 that is placed against a posterior part (e.g. the most posterior part) of the posterior condyle 832 of the femur 801. This is used to set an appropriate level for the planned posterior cut 831 such that an appropriate resection 833 is made. At this point, marks are typically made in the femur 801 using the positioning component 815 to align a subsequent cutting block with a slot in the appropriate position for the planned posterior cut 831. Alternatively, the femoral sizing and positioning instrument could have a slot and the planned posterior cut 831 could be made directly with that instrument as opposed to a separate cutting block.

The next two views 840, 850 depict gap-balancing techniques to determine the location of the posterior cut. In both views 840, 850 a technique is used in which a proximal tibia cut 842, 852 is made at an appropriate angle, depth and position using one or more instruments beyond the scope of this disclosure. The knee can be placed in full extension and the distance between the distal femoral cut 803 on the femur 801 and the proximal tibia cut 842, 852 can be measured using a standard technique, trialing different sizes as needed until the tension in the surrounding soft tissues is felt to be appropriate. The knee can then be placed in flexion as per view 800, and the soft tissues retensioned appropriately.

In one gap-balancing technique depicted in the second view 840, a block 844 matching the distance previously measured in extension 843 can be placed. This block can be placed against the cut surface of the tibia 842, which serves as a posterior landmark, and the location for the ideal posterior cut 841 can be determined such that an appropriate amount of bone can be removed from the posterior condyle 832 of the femur 801 to match the flexion and extension gaps.

In an alternative gap-balancing technique depicted in the third view 850, a stylus 854 can be attached to the posterior component 813 to match the distance previously measured in extension 853. This stylus 854 can be placed against the cut surface of the tibia 852, which serves as a posterior landmark, and the location for the ideal posterior cut 851 can be determined such that an appropriate amount of bone can be removed from the posterior condyle 832 of the femur 801 to match the flexion and extension gaps.

These are just a few representative ways that various landmarks on one or more of the femur 801 and tibia 802 can be used to define a level for the ideal posterior cut in order to optimize the flexion and extension balance. Numerous other instruments can be designed to use the one or more landmarks on the femur and/or the tibia to set a level of a posterior cut. With generally all of the current gap-balancing techniques, focusing primarily on the level of the posterior cut leaves any mismatch to be addressed anteriorly as previously shown in FIG. 5. Adding the ability to also include an anterior landmark into a sizing and positioning instrument as per the embodiments of the present disclosure can instead help a surgeon split any mismatch between the anterior and posterior cuts as shown in FIG. 6.

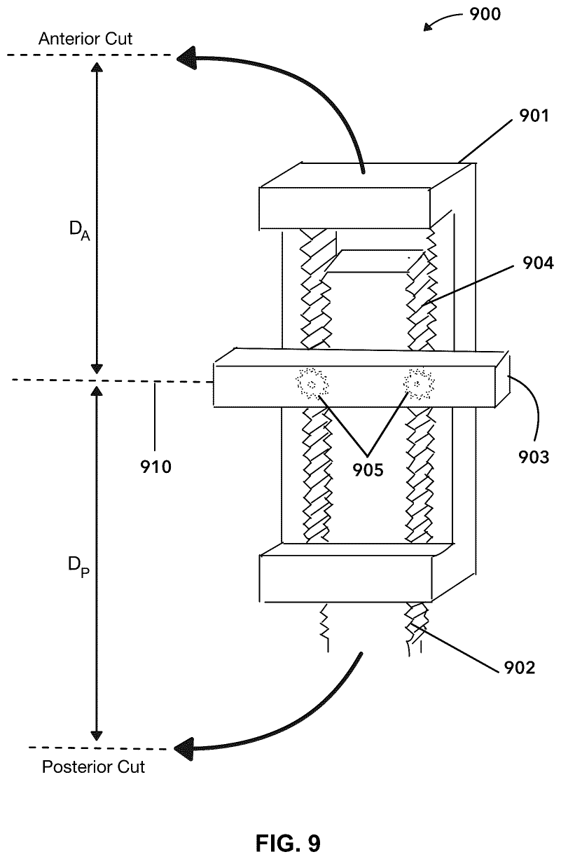

FIG. 9 is a view of the back of a hybrid referencing device 900, according to one embodiment of the present disclosure, and illustrating one embodiment of a mechanism by which the position of a positioning component 903 can be related to both an anterior referencing component 901 and a posterior referencing component 902, and which allows the positioning component 903 to be a variable distance from both an anterior and posterior referencing point on a femur. A position of the anterior referencing component 901 can be related to a position of an anterior landmark. Stated otherwise, the position of the anterior referencing component 901 can be adjusted according to detection of one or more anterior landmarks (e.g., anterior cortex of distal femur) such as using a stylus, as previously described. A position of the posterior referencing component 902 can be related to the position of a posterior landmark. Stated otherwise, the position of the posterior referencing component 902 can be adjusted according to detection of one or more posterior landmarks (e.g., posterior condyles of distal femur) as previously described. A distance from a reference level 910 set by the positioning component 903 to a level of the anterior cut is represented by D_{A} and a distance from the reference level 910 set by the positioning component 903 to a level of the posterior cut is represented by D_{P}.

The hybrid referencing device 900 can include one or more gears 905 that can be rotatably coupled or attached to the positioning component 903. The one or more gears 905 can engage teeth 904 on both the anterior referencing component 901 and the posterior referencing component 902. For example, the hybrid referencing device 900 can include a rack and pinion mechanism to moveably couple the anterior referencing component 901, the posterior referencing component 902, and the positioning component 903. In the illustrated example of FIG. 9, the referencing device 900 can include other mechanisms besides gears, including but not limited to a wheel, a polygon, a toothed mechanism, one or more bars, a chain, and a rack and pinion mechanism.

Those skilled in the art will recognize that if the positioning component 903 is used as a stationary frame of reference, moving the anterior referencing component 901 in an upward direction will necessarily cause a corresponding movement of the posterior referencing component 902 in a downward direction. Because of this, the positioning component 903 does not have a fixed distance from either an anterior reference point or a posterior reference point, but instead has a variable distance in both an anterior and a posterior direction. Stated otherwise, D_{A} and D_{P} are variable according to positioning of the anterior referencing component 901 and the posterior referencing component 902, and not fixed distances.

In this embodiment, the one or more gears 905 and the teeth 904 are designed in such a way that the displacement of the anterior referencing component 901 from the positioning component 903 and of the posterior referencing component 902 from the positioning component 903 have an equal ratio. Moving a referencing component 901, 902 a fixed amount in one direction causes the other referencing component 901, 902 to move the same fixed amount in another direction. In one or more alternative embodiments, one or more of: the gear(s) 905, the teeth 904 on the anterior referencing component 901, and the teeth 904 on the posterior referencing component 902, can be designed in such a way that a different ratio of displacement of the positioning component 903 from the anterior referencing component 901 and the posterior referencing component 902 can be used.

In the embodiment of FIG. 9, the posterior referencing component 902 is shown engaging inside the anterior referencing component 901. A shaft of the posterior referencing component 902 nests within and moves with respect to a channel in the anterior referencing component 901. In one or more alternative embodiments, different configurations of the one or more components can be used. In one or more alternative embodiments, a gear can be replaced by any other mechanism that provides similar functionality including, but not limited to: a pulley, a bearing, a chain, a wheel, and a polygon.

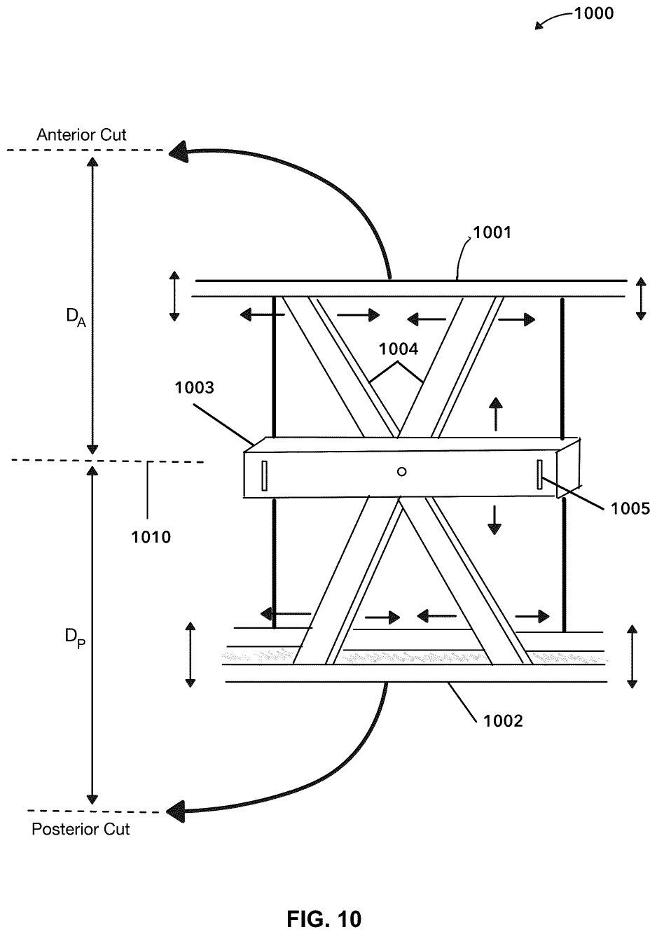

FIG. 10 is a view of the back of a hybrid referencing device 1000, according to one embodiment of the present disclosure, illustrating an alternative embodiment of a mechanism by which the position of a positioning component 1003 can be related to both an anterior referencing component 1001 and a posterior referencing component 1002, and which allows the positioning component 1003 to be a variable distance from both an anterior and posterior referencing point on a femur. In this embodiment of FIG. 10, a position of the anterior referencing component 1001 is related to a position of an anterior landmark as previously described. A position of the posterior referencing component 1002 is related to a position of a posterior landmark as previously described. A distance from the reference level 1010 set by the positioning component 1003 to the level of the anterior cut is represented by D_{A} and a distance from the reference level set by the positioning component 1003 to the level of the posterior cut is represented by D_{P}.

In this embodiment, one or more bars 1004 may be fixed to a positioning component 1003 and allowed to move freely in slots in an anterior referencing component 1001 and a posterior referencing component 1002. As in FIG. 9, those skilled in the art will recognize that if the positioning component 1003 is used as a stationary frame of reference, moving the anterior referencing component 1001 in an upward direction will necessarily cause a corresponding movement of the posterior referencing component 1003 in a downward direction. Because of this, the positioning component 1003 does not remain at a fixed displacement from either an anterior reference point or a posterior reference point, but allows for a variable displacement from both an anterior and a posterior reference. Stated in a different manner, both D_{A} and D_{P} vary as the distance between the anterior referencing component 1001 and the posterior referencing component 1002 varies. This contrasts with existing anterior-referencing systems in which D_{A} does not vary, and from posterior-referencing systems, in which D_{P} does not vary.

In this embodiment of FIG. 10, the amount of displacement between the positioning component 1003 and the anterior referencing component 1001 is equal to the displacement between the positioning component 1003 and the posterior referencing component 1003. In one or more alternative embodiments different ratios between the positioning component 1003, the anterior referencing component 1001 and the posterior referencing component 1003 can be obtained by changing one or more of: the length of the one or more bars 1004, the position of one or more pivot points, the relative lengths of one or more segments on the one or more bars 1004, and attachment points of the one or more bars 1004. Stated otherwise, the ration can be a fixed 1:1 ratio, a fixed ration other than 1:1. In other embodiments, the ratio can be a variable ratio.

The one or more bars 1004 are shown in FIG. 10 as being fixed in position on a positioning component 1003 and allowed to freely move in an anterior referencing component 1001 and a posterior referencing component 1003. In one or more alternative embodiments, the one or more bars 1004 can be fixed or allowed to freely move in any part of the referencing device 1000.

In the embodiment of FIG. 10, because the one or more bars 1004 are only attached to the positioning component 1003 at a single pivot point, in order to keep the positioning component 1003 from rotating around the pivot point, one or more mechanisms 1005 that limit the rotation of the positioning component can be used, which allow for translation but not rotation. In a similar manner, in alternative embodiments, any mechanism which constrains movement of any component in at least one degree of freedom can be utilized.

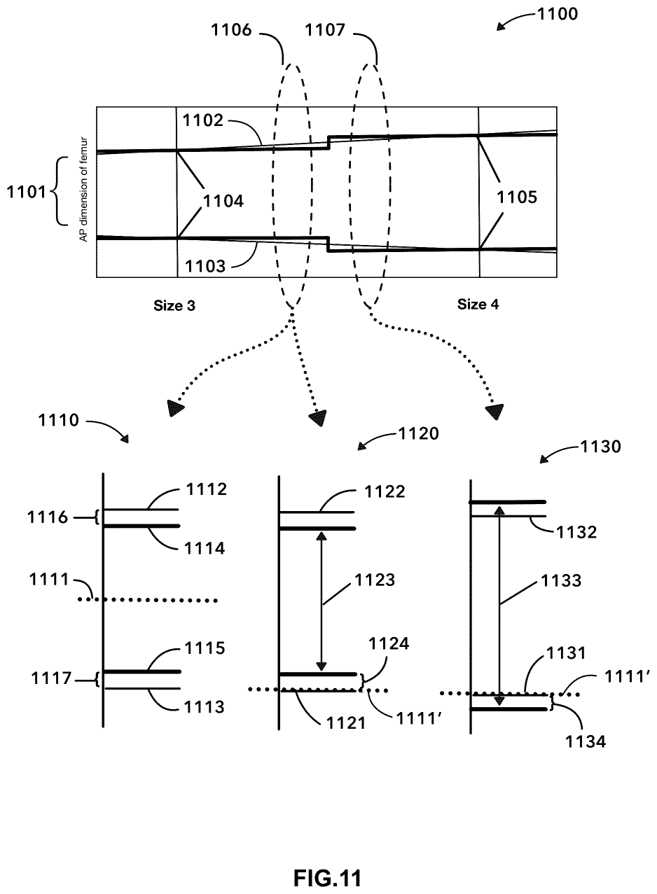

FIG. 11 illustrates a representation of how different frames of reference can be used conceptually to implement concepts from the present disclosure in different embodiments. The graph 1100 at the top is similar to FIG. 6. The y-axis 1101 represents a position of an anterior cut 1102 and a posterior cut 1103 on a femur based on respective landmarks. Lines representing the locations of an ideal anterior cut 1102 and an ideal posterior cut 1103 are shown, with smaller femurs represented toward the left and larger femurs represented toward the right. As per the previous description, there will be patients in whom their anatomy will match an available size. In this example, this match to an available size could be a size 3 1104 or a size 4 1104. For femurs of these sizes, anterior-referencing, posterior-referencing, and hybrid-referencing will all lead to the planned anterior and posterior cuts being in the same place as the ideal anterior and posterior cuts, as there is no mismatch between the patient's anatomy and the AP dimension of the chosen size component. In between sizes, there will be situations 1106 where a size is chosen that is smaller than a patient's anatomy and other situations 1107 where a size is chosen that is larger than a patient's anatomy.

The underlying mathematics (see e.g., FIG. 6) have largely followed the diagram 1110 on the bottom left. In this diagram 1110, a reference point 1111 between an ideal anterior cut 1112 and an ideal posterior cut 1113 is identified. Based on a size chosen by a surgeon, a proposed anterior cut 1114 and proposed posterior cut 1115 are determined such that the distance between the two cuts matches the size of the chosen component, and any mismatch is split between the anterior part of the femur 1116 and the posterior part of the femur 1117.

An alternative mathematical representation of the same situation is shown in a second diagram 1120. In this diagram 1120, the mathematical reference point 1111′ is instead chosen to be the ideal posterior cut 1121. Because an anterior landmark is identified so the entire anterior-posterior dimension of the femur is known, and because the size of the chosen component 1123 is known, a displacement 1124 for the planned posterior cut can be determined in relation to the ideal posterior cut 1121. Similarly, the planned anterior cut is displaced from the ideal anterior cut 1122. Because the size of the component is smaller than the distance between the ideal anterior cut 1122 and the ideal posterior cut 1121, the planned anterior cut will be posterior to the ideal anterior cut 1122.

A third diagram 1130 is similar to the second diagram 1120 where the reference point 1111′ is chosen to be the level of an ideal posterior cut 1131. In this third diagram 1130 a larger size component is chosen with a larger AP dimension 1133. In this case, the planned posterior cut is posteriorly displaced 1134 from the ideal posterior cut 1131. Because the size of the component is larger than the distance between the ideal anterior cut 1132 and the ideal posterior cut 1131, the planned anterior cut will be anterior to the ideal anterior cut 1132.

As related to the graph 1100 above, the amount of displacement can gradually change as the AP dimension 1101 of a femur changes. For example, in a situation where the anatomy matches a size, such as at 1104, there will be no displacement from a posterior-referencing technique. In a situation where the anatomy is slightly larger than a size, such as at 1106, there will be an increasing anterior displacement 1124 based on the relation between the anterior reference point and the posterior reference point. In a situation where the anatomy is slightly smaller than a size, such as at 1107, when a larger size might be chosen, there is a posterior displacement 1134 from a posterior-referencing technique. This posterior displacement will gradually decrease up to the situation where the anatomy matches the next larger size, such as at 1105, where the displacement will once again be zero as there is no mismatch.

Given this, the same outcome of a hybrid-referencing system can be mathematically shown to be equivalent to a posterior-referencing system with automatic displacement based upon additional reference to an anterior landmark. In a similar manner, the same outcome of a hybrid-referencing system can be mathematically shown to be equivalent to an anterior-referencing system with automatic displacement based upon additional reference to a posterior landmark.

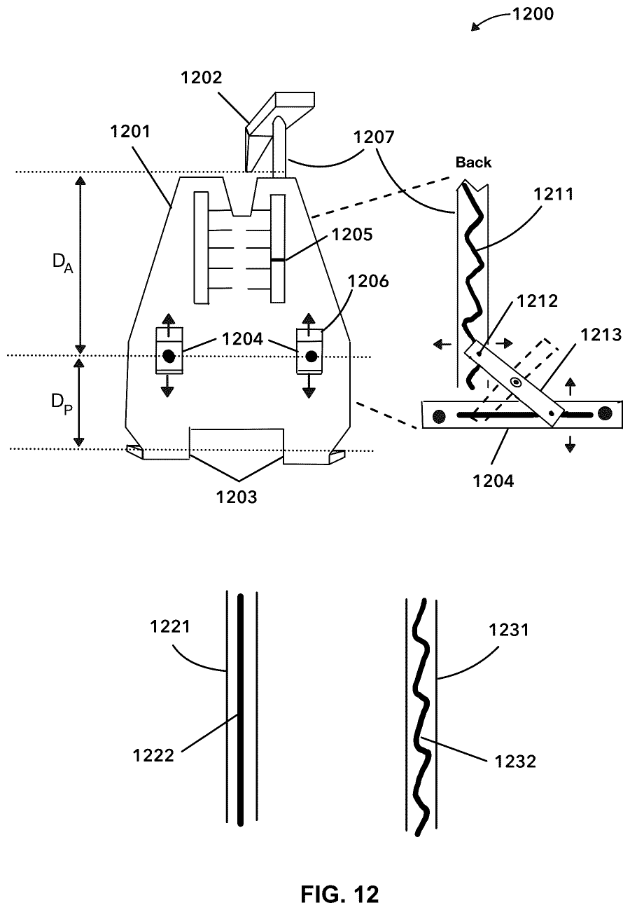

FIG. 12 depicts a hybrid referencing device 1200, according to an embodiment of the present disclosure, and taking advantage of the mathematical equivalence described in FIG. 11. The hybrid referencing device 1200 may in essence modify a posterior referencing system to also take into account an anterior reference (e.g., anterior landmarks). A posterior referencing component 1201 of the hybrid referencing device 1200 may be similar to referencing devices existing on the market in posterior-referencing systems. The referencing device 1200, and more particularly the posterior referencing component 1201, includes posterior feet 1203 to reference a posterior landmark. The referencing device 1200 also includes a stylus 1202 to reference an anterior landmark. There are guides 1204 (e.g., holes) to mark a position based on a fixed displacement from the feet 1203 (e.g., where the feet 1203 detect, engage, or otherwise interface with a posterior landmark).

In current systems (e.g., other existing systems), there can be a way for a surgeon to manually change the chosen position of the guides 1204 including but not limited to one or more of: additional guides (e.g., holes) with anterior or posterior displacement, and a dial or other mechanism to manually shift the position of the guides anterior or posterior.

However, in the hybrid referencing device 1200 of FIG. 12, the guides 1204 (e.g., collectively a positioning component) used to mark a desired position for a subsequent cut block are automatically moved anterior or posterior in a displacement window 1206 based on the position of the stylus 1202 which is attached to a stylus rod 1207 which can move up and down relative to and within the posterior referencing component 1201. This stylus rod 1207 can be used to show or otherwise indicate an appropriate size 1205 for a given patient's anatomy.

One method of accomplishing this automatic adjustment of displacement in the displacement window 1206 is demonstrated by the view of a groove 1211 on the back of the stylus rod 1207. As the stylus is moved up or down, a pin 1212 in the stylus 1207 is moved side-to-side. A bar 1213 attached to this pin 1212 passes through a pivot point and changes the side-to-side movement at one end into an anterior-posterior displacement (e.g., up and down movement) of the positioning component 1204. The geometry of the groove can be determined such that there is no displacement when the size of a particular femur matches an available component size. The positioning component 1204 can be moved anterior when the femur is “downsized” (i.e., a smaller size is chosen than is measured) to split the mismatch anteriorly and posteriorly. The positioning component 1204 can be moved posteriorly when the femur is “upsized” (i.e., a larger size is chosen than is measured). The options illustrated can, for example, correspond to locations 1104, 1105, 1106 and 1107 on FIG. 11. In this manner, the device 1200 of FIG. 12 can implement mechanically what is demonstrated mathematically in FIG. 11.

In addition to enabling a hybrid-technique, by changing the stylus rod 1207 in the same posterior referencing component 1201 of a referencing device 1200, a surgeon has the option to mimic any of the standard techniques shown in FIG. 5, including anterior-referencing and posterior-referencing. For example, if a straight groove 1222 is on the back of the stylus rod 1221, there will be no displacement at any position (i.e., D_{P} will not change but will be a fixed displacement). This effectively transforms this referencing device 1200 into a posterior-referencing system where there is no displacement in relation to a posterior landmark (see graph 520 of FIG. 5). This might be useful in a patient where a surgeon is primarily concerned about balancing flexion-extension and is willing to accept more mismatch anteriorly.

Alternatively, by having an alternate pattern of grooves 1232 in an alternative stylus rod 1231, the amount of displacement can be changed such that there is no mismatch anteriorly (i.e., D_{A} will not change but be a fixed displacement), effectively turning this embodiment into an anterior-referencing system (see graph 510 in FIG. 5). This might be useful in a patient where a surgeon is primarily concerned about avoiding notching or patellofemoral overstuffing and is willing to accept more potential flexion-extension imbalance.

Overall, a feature of the hybrid referencing device 1200 of FIG. 12 is that this embodiment allows for a single set of instrumentation to be used for various techniques including, but not limited to, one or more of: anterior-referencing, posterior-referencing and hybrid-referencing. A surgeon can decide during a case to stay with hybrid-referencing, but if there is osteoporotic bone, for example, they may choose to swap out the hybrid stylus rod 1207 to an anterior-referencing rod 1231 to ensure there is no notching. This gives a surgeon maximum flexibility in matching each procedure to each individual patient. For manufacturers, the flexibility feature allows them to offer a single system to all surgeons, whether they prefer anterior-referencing, posterior-referencing or hybrid-referencing, and whether they prefer measured resection techniques or gap-balancing techniques. Further, the flexibility of the hybrid referencing device 1200 of FIG. 12 allows use of the device with existing sets of cutting blocks, such that a femoral sizing and positioning system can be retrofitted with just a hybrid referencing device without needing to replace sets of cutting blocks.

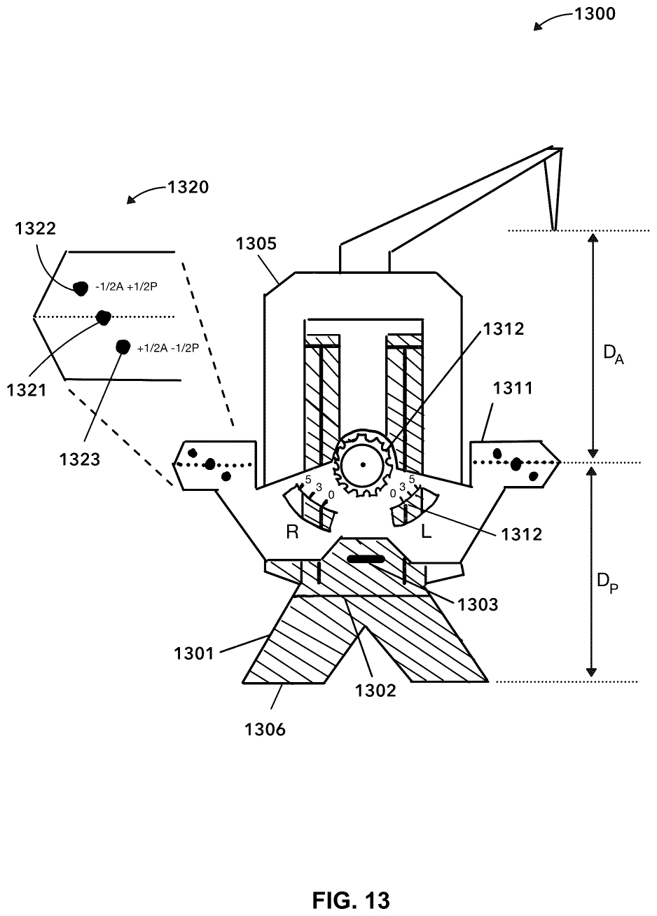

FIG. 13 is hybrid referencing device 1300, according to another embodiment of the present disclosure, and showing a few additional features that are possible or available. The hybrid referencing device 1300 includes an anterior referencing component 1305, a posterior referencing component 1306, and a positioning component 1311.

As shown in FIG. 8 and described above with reference to the same, there are various ways of determining a posterior reference point. In view 830, a landmark on the posterior femur can be identified. This can be accomplished by the posterior stylus, or “feet” 1301, shown on the device 1300 of FIG. 13. The feet can be placed against the posterior condyles of the femur. These can alternatively also include various angles and shapes.

An example of a gap-balancing technique using a block is also shown in view 840 of FIG. 8. This technique can be accomplished using the device 1300 of FIG. 13 by removing the feet 1301 and resting the resulting flat edge 1302 of the posterior referencing component on the sizer block 844. When tensioned, this allows for a posterior reference point related to the proximal tibia 842 to be used in conjunction with an anterior landmark to effect a hybrid position and split any mismatch both anteriorly and posteriorly.

Another example of a gap-balancing technique is shown in view 850 using an instrument 854 (e.g., stylus) of a fixed or variable depth which is attached to the posterior referencing component. This technique can be accomplished using the device 1300 by removing the feet 1301 and a slot 1303 or other method for attaching an instrument 854 can be used. This configuration of the device 1300 allows for a posterior referencing point on the tibia 852 to be used in conjunction with an anterior landmark to effect a hybrid position.

The device 1300 also allows, as shown in FIG. 13, for a surgeon to position the femoral component at varying degrees of internal or external rotation. A knob 1312 can be loosened and the positioning component 1311 can be rotated to a desired angle to make or otherwise guide marking holes. There are additional ways of changing rotation not shown. Importantly, this works in conjunction with the hybrid-referencing technique by referencing both an anterior and a posterior landmark, with distances D_{A}, D_{P} as previously defined.

While one method of fine-tuning positioning to mimic an anterior-referencing system or a posterior-referencing technique with a hybrid-referencing device 1200 is shown in FIG. 12, another method is demonstrated by the closeup view 1320 here in FIG. 13. To use the referencing device 1300 with a hybrid-referencing technique as described herein, a surgeon can use a standard central marking hole 1321. This could be used for femurs that match a size exactly. This could also be used for a femur that is midway between sizes, which would split any mismatch between the anterior and posterior cuts in a hybrid-referencing technique

As in prior descriptions, there may be times when a surgeon is more concerned about matching an ideal anterior cut more exactly at the expense of posterior mismatch. This might be the case, for example, with osteoporotic bone where a notch may weaken the bone too much. In this case, if a surgeon was between sizes (for example, 3.5) and wanted to use a smaller femur in an anterior-referencing equivalent mode (for example, size 3), they would use the hole marked −½A 1322. Using this hole will move the planned anterior and posterior cuts anterior to help avoid creating a notch. If a surgeon was between sizes (for example, 3.5) and wanted to use a larger size (for example, size 4), they would use the hole marked +½A 1323. Using this hole will move planned anterior and cuts posteriorly, again matching the anterior landmark at the potential expense of introducing more mismatch posteriorly. Overall, by using the holes marked ‘A’ for in-between sizes, a surgeon could also use a hybrid-refencing sizing and positioning guide in an anterior-referencing mode.

Alternatively, a surgeon may want to use this embodiment in posterior-referencing equivalent mode. This might be useful in a high-demand patient with strong bone in whom matching the flexion-extension gap might be felt to be most important. In this case, if the sizing and positioning guide showed that a femur was “between” sizes, a surgeon could use the hole marked +½P 1322 if they wanted to upsize the component (i.e., use a size 4 in a size 3.5 femur). They could use the hole marked −½P 1323 if they wanted to downsize the component (i.e., use a size 3 for a size 3.5 femur). Overall, by using the holes marked ‘P’ for in-between sizes, a surgeon could also use a hybrid-referencing sizing and positioning guide in a posterior-referencing mode.

### Example Embodiments

The following are non-limiting examples of embodiments of the present disclosure.

Example 1. A hybrid referencing device for knee arthroplasty, comprising: an anterior referencing component to obtain a position of one or more anterior landmarks related to an anterior part of a distal femur; a posterior referencing component to obtain a position of one or more posterior landmarks related to one or more of a posterior part of the distal femur and a proximal part of a tibia; and a positioning component coupled to, and moveable relative to, the anterior referencing component and the posterior referencing component to provide a position related to and variable from both: the one or more anterior landmarks; and the one or more posterior landmarks. The posterior referencing component can be coupled to and moveable relative to the anterior referencing component.

Example 2. The device of Example 1, wherein the positioning component is mechanically linked to the anterior referencing component and the posterior referencing component by a mechanism comprising, but not limited to, one or more of the following: a gear, a wheel, a polygon, a toothed mechanism, one or more bars, a chain, and a rack and pinion gear mechanism.

Example 3. The device of Example 1, wherein, during use of the positioning component as a frame of reference at the distal surface of the distal surface of the distal femur, a displacement of the anterior referencing component in one direction causes a displacement of the posterior referencing component in an opposite direction in a ratio comprising one or more of: a fixed 1:1 ratio; a fixed ratio other than 1:1; and a variable ratio.

Example 4. The device of Example 1, wherein an initial location of the positioning component in relation to the posterior referencing component is defined, and additional displacement of the positioning component corresponds to a change to a location of the anterior referencing component.

Example 5. The device of Example 1, wherein an initial location of the positioning component in relation to the anterior referencing component is defined, and additional displacement of the positioning component corresponds to a change to a location of the posterior referencing component.

Example 6. The device of Example 1, further comprising one or more markings to indicate available sizes of a femoral component, wherein the one or more markings are to allow a user to choose an appropriate size for the femoral component.

Example 7. The device of Example 1, wherein the positioning component includes a feature to enable marking one or more locations on the distal femur, the feature comprising, but not limited to, one or more of the following: a drill hole, a pin hole, a punch guide, a saw guide, a notch and a defined surface for marking.

Example 8. The device of Example 1, wherein the anterior referencing component comprises a stylus to extend to interface with the one or more anterior landmarks.

Example 9. The device of Example 1, wherein the posterior referencing component comprises one or more features to interface with the one or more posterior landmarks, the one or more features including, but not limited to, one or more of: posterior feet to engage (e.g., touch, interface with) one or more posterior femoral condyles of the femur; a surface of the posterior referencing component to rest on a sizing block, the sizing block to engage (e.g., touch, interface with) a surface of the tibia; and a stylus attached to the posterior referencing component, the stylus to engage (e.g., touch, interface with) a surface of the tibia.

Example 10. The device of Example 1, wherein the anterior referencing component comprises an anterior referencing rod which can freely move in the posterior referencing component, the anterior referencing rod mechanically linked to the positioning component such that movement of the anterior referencing rod in relation to the posterior referencing component causes a related movement of the positioning component in relation to the posterior referencing component.

Example 11. The device of Example 10, wherein the configuration of the mechanical linkage between the anterior referencing rod and the positioning component is modifiable, such that the location of the positioning component causes the hybrid referencing device to be configured to be used for one or more of: an anterior-referencing technique, where the location of the positioning component is such that an anterior cut on the femur remains a specific distance from the anterior landmark; a posterior-referencing technique, where the location of the positioning component is such that a posterior cut on the femur remains a specific distance from the posterior landmark; and a hybrid-referencing technique, where the location of the positioning component is such that the distance from an anterior cut on the femur to the anterior landmark and the distance from a posterior cut on the femur to the posterior landmark both vary.

Example 12. A femoral referencing device for knee arthroplasty, comprising: an anterior referencing component configured to engage (e.g., abut, touch, interface with) an anterior side of a distal end of a femur to interface one or more anterior landmarks on an anterior surface of a distal end of a femur; a posterior referencing component configured to engage (e.g., abut, touch, interface with) a posterior side of the distal end of the femur to interface one or more posterior landmarks at one or more of a posterior surface of the distal end of the femur and a proximal part of a tibia, the posterior referencing component coupled to and moveable relative to the anterior referencing component; and a positioning component coupled to, and moveable relative to, the anterior referencing component and the posterior referencing component to provide a position related to and variable from both the one or more anterior landmarks and the one or more posterior landmarks.

Example 13. The device of Example 12, wherein a first portion of the anterior referencing component is coupled to a first portion of the posterior referencing component; a second portion of the anterior referencing component is configured to engage the one or more anterior landmarks; and a second portion of the posterior referencing component is configured to engage the one or more posterior landmarks.

Example 14. The device of Examples 13, wherein the positioning component is coupled to the anterior referencing component and the posterior referencing component between the second portion of the anterior referencing component and the second portion of the posterior referencing component.

Example 15. The device of Example 12, wherein the positioning component is mechanically linked to the anterior referencing component and the posterior referencing component by a moveable mechanism.

Example 16. The device of Example 15, wherein the moveable mechanism comprises one or more of the following: a gear, a wheel, a polygon, a toothed mechanism, one or more bars, a chain, and a rack and pinion gear mechanism.

Example 17. The device of Example 12, wherein, during use of the positioning component as a frame of reference at the distal surface of the distal surface of the distal femur, a displacement of the anterior referencing component in one direction causes a displacement of the posterior referencing component in an opposite direction in a ratio comprising one or more of: a fixed 1:1 ratio; a fixed ratio other than 1:1; and a variable ratio.

Example 18. The device of Example 12, wherein an initial location of the positioning component in relation to the posterior referencing component is defined, and additional displacement of the positioning component is according to a change to a location of the anterior referencing component.

Example 19. The device of Example 12, wherein an initial location of the positioning component in relation to the anterior referencing component is defined, and additional displacement of the positioning component is according to a change to a location of the posterior referencing component.

Example 20. The device of Example 12, wherein, during use of the positioning component as a frame of reference at the distal surface of the distal surface of the distal femur, a displacement of the anterior referencing component in one direction causes a displacement of the posterior referencing component in an opposite direction.

Example 21. The device of Example 12, wherein an initial location of the positioning component is a displacement a distance D_{P} from the posterior referencing component and a displacement D_{A} from the anterior referencing component, and wherein a movement of the posterior referencing component relative to the anterior referencing component results in a new location of the positioning component a distance D′_{P} from the posterior referencing component and a displacement D′_{A} from the anterior referencing component.

Example 22. The device of Example 12, wherein the anterior referencing component comprises: a main body (e.g., a housing, a cutting block) having a longitudinal axis to extend parallel to an anteroposterior (AP) axis of the distal femur; and a stylus coupled at a first end to the main body and extending transverse to the longitudinal axis of the main body to interface at a second end with the one or more anterior landmarks.

Example 23. The device of Example 12, wherein the anterior referencing component comprises a stylus to extend to interface with the one or more anterior landmarks.

Example 24. The device of Example 12, wherein the posterior referencing component comprises: a main body having a longitudinal axis to extend parallel to an anteroposterior (AP) axis of the distal femur; and one or more tabs to extend transverse to the longitudinal axis of the main body to interface the one or more posterior landmarks.

Example 25. The device of Example 12, wherein the posterior referencing component comprises one or more tabs to extend to interface the one or more posterior landmarks.

Example 26. The device of Example 12, wherein an amount of displacement (e.g., a distance) D_{A} from the anterior referencing component to the positioning component and the amount of displacement (e.g., a distance) D_{P} from the posterior referencing component to the positioning component are one of the following: equal; any fixed ratio other than 1:1; and any variable ratio.

Example 27. A hybrid femoral referencing device for femoral component sizing and/or positioning in knee arthroplasty, comprising: an anterior referencing component to engage one or more anterior landmarks at an anterior of a distal femur; a posterior referencing component to engage one or more posterior landmarks at one or more of a posterior of the distal femur or a proximal end of a tibia, the posterior referencing component coupled to and moveable relative to the anterior referencing component; and a positioning component moveably coupled to the anterior referencing component and the posterior referencing component to provide a position according to and variable from both: the one or more anterior landmarks; and the one or more posterior landmarks.

Example 28. A femoral referencing device (e.g., a sizing and/or positioning device) for femoral component sizing and/or positioning in knee arthroplasty, comprising: an anterior referencing component to determine, detect, identify, or otherwise obtain a position of one or more anterior landmarks related to or at an anterior part (e.g. surface, side, portion) of a distal femur; a posterior referencing component to obtain a position of one or more posterior landmarks related to one or more of a posterior part (e.g. surface, side, portion) of the distal femur and a proximal part of a tibia, the posterior referencing component coupled to and moveable relative to the anterior referencing component; and a positioning component moveably (and medially) coupled to the anterior referencing component and the posterior referencing component to provide a position related to and variable from both: the one or more anterior landmarks; and the one or more posterior landmarks.

Example 29. The device of Example 28, wherein the positioning component is mechanically linked to the anterior referencing component and the posterior referencing component by a moveable mechanism comprising one or more of the following: a gear, a wheel, a polygon, a toothed mechanism, one or more bars, and a chain.

Example 30. The device of Example 28, wherein the positioning component is mechanically liked to and movable relative to the anterior referencing component and the posterior referencing component by a rack and pinion gear mechanism.

Example 31. The device of Example 28, wherein the anterior referencing component and the posterior referencing component coupled by one or more slides (e.g., dovetail slide, boxway slide, sleeve bearings, linear ball slide/carriage) to be moveable relative to each other by sliding.

Example 32. The device of Example 31, wherein the one or more slides each comprise: a protrusion; and a corresponding groove.

Example 33. The device of Example 31, wherein the one or more slides each comprise: a rail; and a carriage.

Example 34. The device of Example 28, wherein the anterior referencing component comprises a channel to engage a protrusion extending from a bar of a linkage to adjust the positioning component relative to the anterior referencing component and posterior referencing component.

Example 35. The device of Example 28, wherein, during use of the positioning component as a frame of reference at the distal surface of the distal surface of the distal femur, a displacement of the anterior referencing component in one direction causes a displacement of the posterior referencing component in an opposite direction.

Example 36. The device of Example 28, wherein an initial location of the positioning component is a displacement a distance D_{P} from the posterior referencing component and a displacement D_{A} from the anterior referencing component, and wherein a movement of the posterior referencing component relative to the anterior referencing component results in a new location of the positioning component a distance D′_{P} from the posterior referencing component and a displacement D′_{A} from the anterior referencing component.

Example 37. The device of Example 28, wherein the amount of displacement D_{A} from the anterior referencing component to the positioning component and the amount of displacement D_{P} from the posterior referencing component to the positioning component are one of the following: equal; any fixed ratio other than 1:1; and any variable ratio.

Example 38. The device of Example 28, further comprising markings to indicate available sizes of a femoral component, wherein the markings are to allow a user to choose an appropriate size for the femoral component.

Example 39. The device of Example 28, wherein the positioning component includes holes to guide marking one or more locations on the distal femur.

Example 40. The device of Example 1, wherein the anterior referencing component comprises a stylus to extend to interface the one or more anterior landmarks.

Example 41. The device of Example 1, wherein the anterior referencing component comprises: a main body (e.g. a housing, a cutting block) having a longitudinal axis to extend parallel to an anteroposterior (AP) axis of the distal femur; and a stylus coupled at a first end to the main body and extending transverse to the longitudinal axis of the main body to interface at a second end with the one or more anterior landmarks.

Example 42. The device of Example 28, wherein the posterior referencing component comprises one or more tabs to extend to interface the one or more posterior landmarks.

Example 43. The device of Example 28, wherein the posterior referencing component comprises: a main body (e.g. a housing, a cutting block) having a longitudinal axis to extend parallel to an anteroposterior (AP) axis of the distal femur; and on or more tabs to extend transverse to the longitudinal axis of the main body to interface the one or more posterior landmarks.

A referencing device, or a sizing and/or positioning device, as referred to herein, can be a generic for the device or instrument used to choose a size for a femoral component and mark a position on the femur to make appropriate cuts.

An anterior landmark, as referred to herein, can be an anatomic point used for reference for the anterior cut. Generally, the anterior cortex of the femur is an anterior landmark but other landmarks can be used.

A posterior landmark, as referred to herein, can be an anatomic point used for reference for the posterior cut. Generally, either the posterior condyle of the femur or a proximal part of the tibia are posterior landmarks, but other landmarks can be used.

An anterior referencing component, as referred to herein, is a part of a device or instrument that is used to obtain get an anterior referencing point.

An anterior stylus can be attached to, part of, or integrated with the anterior referencing component and used to refer to the anterior landmark to set a known distance from the landmark to the anterior referencing component.

A posterior referencing component, as referred to herein, can be a part of a device or instrument used to obtain a posterior referencing point

A positioning component, as referred to herein, is a part of an instrument or device that is used to set a position for planned cuts.

The phraseology and terminology used herein is for the purpose of description and should not be regarded as limiting. Any references to implementations or elements or acts of the systems and methods herein referred to in the singular may also embrace implementations including a plurality of these elements, and any references in plural to any implementation or element or act herein may also embrace implementations including only a single element. Any implementation disclosed herein may be combined with any other implementation or embodiment.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

In some cases, well-known features, structures, or operations are not shown or described in detail. Furthermore, the described features, structures, or operations may be combined in any suitable manner in one or more embodiments. It will also be readily understood that the components of the embodiments as generally described and illustrated in the figures herein could be arranged and designed in a wide variety of different configurations.

Various operational steps, as well as components for carrying out operational steps, may be implemented in alternate ways depending on the particular application or in consideration of any number of cost functions associated with the operation of the systems, e.g., one or more of the steps may be deleted, modified, or combined with other steps.

While the principles of this disclosure have been shown in various embodiments, many modifications of structure, arrangements, proportions, the elements, materials and components, used in practice, which are particularly adapted for a specific environment and operating requirements, may be used without departing from the principles and scope of this disclosure. These and other changes or modifications are intended to be included within the scope of the present disclosure.

The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

The scope of the present invention should, therefore, be determined only by the following claims:

What is claimed is:

1. A hybrid referencing device for knee arthroplasty, comprising:
an anterior referencing component to obtain a position of one or more anterior landmarks related to an anterior part of a distal femur;
a posterior referencing component to obtain a position of one or more posterior landmarks related to one or more of a posterior part of the distal femur and a proximal part of a tibia; and
a positioning component coupled to, and moveable relative to, the anterior referencing component and the posterior referencing component to provide a position related to and variable from both:
the one or more anterior landmarks; and
the one or more posterior landmarks.

2. The device of claim 1, wherein the positioning component is mechanically linked to the anterior referencing component and the posterior referencing component by a mechanism comprising, but not limited to, one or more of the following:
a gear,
a wheel,
a polygon,
a toothed mechanism,
one or more bars,
a chain, and
a rack and pinion gear mechanism.

3. The device of claim 1, wherein, during use of the positioning component as a frame of reference at a distal surface of the distal femur, a displacement of the anterior referencing component in one direction causes a displacement of the posterior referencing component in an opposite direction in a ratio comprising one or more of:
a fixed 1:1 ratio;
a fixed ratio other than 1:1; and
a variable ratio.

4. The device of claim 1, wherein an initial location of the positioning component in relation to the posterior referencing component is defined, and additional displacement of the positioning component corresponds to a change to a location of the anterior referencing component.

5. The device of claim 1, wherein an initial location of the positioning component in relation to the anterior referencing component is defined, and additional displacement of the positioning component corresponds to a change to a location of the posterior referencing component.

6. The device of claim 1, further comprising one or more markings to indicate available sizes of a femoral component, wherein the one or more markings are to allow a user to choose an appropriate size for the femoral component.

7. The device of claim 1, wherein the positioning component includes a feature to enable marking one or more locations on the distal femur, the feature comprising, but not limited to, one or more of the following: a drill hole, a pin hole, a punch guide, a saw guide, a notch and a defined surface for marking.

8. The device of claim 1, wherein the anterior referencing component comprises a stylus to extend to interface with the one or more anterior landmarks.

9. The device of claim 1, wherein the posterior referencing component comprises one or more features to interface with the one or more posterior landmarks, the one or more features including, but not limited to, one or more of:
posterior feet to engage one or more posterior femoral condyles of the femur;
a surface of the posterior referencing component to rest on a sizing block, the sizing block to engage a surface of the tibia; and
a stylus attached to the posterior referencing component, the stylus to engage a surface of the tibia.

10. The device of claim 1, wherein the anterior referencing component comprises an anterior referencing rod which can freely move in the posterior referencing component, the anterior referencing rod mechanically linked to the positioning component such that movement of the anterior referencing rod in relation to the posterior referencing component causes a related movement of the positioning component in relation to the posterior referencing component.

11. The device of claim 10, wherein a configuration of the mechanical linkage between the anterior referencing rod and the positioning component is modifiable, such that a location of the positioning component causes the hybrid referencing device to be configured to be used for one or more of:
an anterior-referencing technique, where the location of the positioning component is such that an anterior cut on the femur remains a specific distance from the one or more anterior landmarks;
a posterior-referencing technique, where the location of the positioning component is such that a posterior cut on the femur remains a specific distance from the one or more posterior landmarks; and
a hybrid-referencing technique, where the location of the positioning component is such that the distance from an anterior cut on the femur to the one or more anterior landmarks and the distance from a posterior cut on the femur to the one or more posterior landmarks both vary.

12. A femoral referencing device for knee arthroplasty, comprising:
an anterior referencing component configured to engage an anterior side of a distal end of a femur to interface one or more anterior landmarks on an anterior surface of a distal end of a femur;
a posterior referencing component configured to engage a posterior side of the distal end of the femur to interface one or more posterior landmarks at one or more of a posterior surface of the distal end of the femur and a proximal part of a tibia, the posterior referencing component coupled to and moveable relative to the anterior referencing component; and
a positioning component coupled to, and moveable relative to, the anterior referencing component and the posterior referencing component to provide a position related to and variable from both the one or more anterior landmarks and the one or more posterior landmarks.

13. The device of claim 12, wherein the positioning component is mechanically linked to the anterior referencing component and the posterior referencing component by a moveable mechanism.

14. The device of claim 12, wherein, during use of the positioning component as a frame of reference at a distal surface of the distal end of the femur, a displacement of the anterior referencing component in one direction causes a displacement of the posterior referencing component in an opposite direction in a ratio comprising one or more of:
a fixed 1:1 ratio;
a fixed ratio other than 1:1; and
a variable ratio.

15. The device of claim 12, wherein an initial location of the positioning component in relation to the posterior referencing component is defined, and additional displacement of the positioning component is according to a change to a location of the anterior referencing component.

16. The device of claim 12, wherein an initial location of the positioning component in relation to the anterior referencing component is defined, and additional displacement of the positioning component is according to a change to a location of the posterior referencing component.

17. The device of claim 12, wherein, during use of the positioning component as a frame of reference at the distal surface of the distal end of the femur, a displacement of the anterior referencing component in one direction causes a displacement of the posterior referencing component in an opposite direction.

18. The device of claim 11, wherein an initial location of the positioning component is a displacement a distance D_{P} from the posterior referencing component and a displacement D_{A} from the anterior referencing component,
wherein a movement of the posterior referencing component relative to the anterior referencing component results in a new location of the positioning component a distance D′_{P} from the posterior referencing component and a displacement D′_{A} from the anterior referencing component.

19. The device of claim 12, wherein the anterior referencing component comprises:
a main body having a longitudinal axis to extend parallel to an anteroposterior (AP) axis of the distal end of the femur; and
a stylus coupled at a first end to the main body and extending transverse to the longitudinal axis of the main body to interface at a second end with the one or more anterior landmarks.

20. The device of claim 12, wherein the posterior referencing component comprises:
a main body having a longitudinal axis to extend parallel to an anteroposterior (AP) axis of the distal end of the femur; and
one or more tabs to extend transverse to the longitudinal axis of the main body to interface the one or more posterior landmarks.
